(19) United States
(12) Patent Application Publication
(10) Pub. No.: US 2026/0201293 A1
(43) Pub. Date: Jul. 16, 2026

(54) GAS EXCHANGE OPTIMIZED WELL PLATE

(71) Applicant: Adam KUHN, Houston, TX (US)

(72) Inventor: Adam KUHN, Houston, TX (US)

(21) Appl. No.: 19/022,564

(22) Filed: Jan. 15, 2025

(51) Int. Cl. C12M 1/32; C12M 1/00; C12M 1/12; C12M 3/00

(52) U.S. Cl. CPC C12M 23/12; C12M 23/34; C12M 23/38; C12M 23/42; C12M 23/58; C12M 25/14

(57) ABSTRACT

A multi-well cell culture device for optimizing gas exchange and nutrient availability in cell culture, comprising a Gas Exchange Optimized Well Plate (GEOWP) base, the GEOWP base comprising a plurality of alternating cell culture wells and reservoir wells, each of the plurality of cell culture wells having an elevated cell culture stage to culture adherent cells and a first plurality of openings above the elevated cell culture stage to be connected to a corresponding adjacent reservoir well which stores additional cell culture medium; a GEOWP lid having a plurality of cylindrical protrusions to seal the plurality of reservoir wells; and a GEOWP insert which fits between the GEOWP base and the GEOWP lid, the GEOWP insert having a plurality of downward protrusions to cover the first plurality of openings to disconnect volumes above and below the elevated cell culture stages associated with the plurality of cell culture wells.

### TECHNICAL FIELD

Embodiments of the disclosure generally relate to equipment used to culture cells in a laboratory, and more particularly, to a Gas Exchange Optimized Well Plate (GEOWP) for optimizing gas exchange for adherent cells.

### BACKGROUND

Cell culture is an important tool in life science for growing cells from an animal or a plant in an artificial environment outside their natural environment. The ability to keep somatic cells alive independently of their usual multicellular context has vastly expanded the repertoire of experiments available to researchers. Thus, different cell culture systems range from large plastic dishes in which many cells can be grown easily, to plates of wells which include a plurality of hollow cylinders to hold groups of cells separately, accommodating experiments with multiple varying conditions. In particular, adherent cells play an important role in the bulk of human cell culture applications in cell biology, biochemistry, and cancer research. To culture the adherent cells, the cell culture systems often involve a flat circular surface to which the adherent cells adhere. For example, one or more cylindrical walls extend upwards around the circumference of a circular plate on which the adherent cells sit to hold a cell culture medium. The cell culture medium may be a fluid which provides nutrients and conditions the adherent cells to grow in the artificial environment. Thus, the cell culture medium is often carefully designed for a solution to meet a plurality of demands of culturing different cells. The plurality of demands may include a reduced carbon source to supply the different cells with a substrate for oxidative phosphorylation (energy production), ions to facilitate transmembrane transport, and a bicarbonate buffer to keep a potential of hydrogen (pH) level of the solution stable.

### SUMMARY

The following presents a simplified summary of the disclosed subject matter in order to provide a basic understanding of some aspects of the subject matter disclosed herein. This summary is not an exhaustive overview of the technology disclosed herein. It is not intended to identify key or critical elements of the disclosed subject matter or to delineate the scope of the disclosed subject matter. Its sole purpose is to present some concepts in a simplified form as a prelude to the more detailed description that is discussed later.

In one or more embodiments, the present invention provides a multi-well cell culture device for optimizing gas exchange and nutrient availability in a cell culture. The multi-well cell culture device includes a Gas Exchange Optimized Well Plate (GEOWP) base. In particular, the GEOWP base is made of plastic. The GEOWP base includes a plurality of alternating cell culture wells and reservoir wells. Each of the plurality of cell culture wells has an elevated cell culture stage to culture adherent cells. Each of the plurality of cell culture wells comprises two concentric hollow cylinders. Each of the plurality of reservoir wells comprises a hollow cylinder which is configured to store the additional cell culture medium in an airtight fashion and connected to the plurality of cell culture wells through a channel. The GEOWP base further includes a first plurality of openings above the elevated cell culture stage to be connected to a corresponding adjacent reservoir well which stores additional cell culture medium. The multi-well cell culture device further includes a GEOWP lid having a plurality of cylindrical protrusions to seal the plurality of reservoir wells. The multi-well cell culture device further includes a GEOWP insert which fits between the GEOWP base and the GEOWP lid. The GEOWP insert has a plurality of downward protrusions to cover the first plurality of openings associated with the plurality of cell culture wells to disconnect volumes above and below the elevated cell culture stages associated with the plurality of cell culture wells. Each of the plurality of cell culture wells has a second plurality of openings below the elevated cell culture stage. A volume above the elevated cell culture stage is linked to a volume below the elevated cell culture stage via the first plurality of openings above the elevated cell culture stage and the second plurality of openings below the elevated cell culture stage. The plurality of reservoir wells uses multiple static components to optimize gas exchange in the culturing of adherent cells. A cell monolayer of the adherent cells which adhere to the elevated cell culture stage is raised to within a predetermined distance from the surface of the cell culture medium to avoid inducing hypoxia in the cells. For example, the cell monolayer of the adherent cells resides only about 2 millimeters (mm) from the surface of the cell culture medium.

Other aspects and advantages of the claimed subject matter will be apparent from the following description and the appended claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 illustrates a Gas Exchange Optimized Well Plate (GEOWP) in accordance with one or more embodiments.

FIG. 2A illustrates a top-angle view of a GEOWP base in accordance with one or more embodiments.

FIG. 2B illustrates a top-front angle view of a GEOWP base in accordance with one or more embodiments.

FIG. 2C illustrates a front view cross-section view of a GEOWP base in accordance with one or more embodiments.

FIG. 3A illustrates a top-angle view of a GEOWP insert in accordance with one or more embodiments.

FIG. 3B illustrates a top-front cross-section view of a GEOWP insert in accordance with one or more embodiments.

FIG. 3C illustrates a front angle cross-section view of a GEOWP insert in accordance with one or more embodiments.

FIG. 4A illustrates a bottom-angle view of a GEOWP lid in accordance with one or more embodiments.

FIG. 4B illustrates a bottom angle cross-section view of a GEOWP lid in accordance with one or more embodiments.

While certain embodiments will be described in connection with the illustrative embodiments shown herein, the subject matter of the present disclosure is not limited to those embodiments. On the contrary, all alternatives, modifications, and equivalents are included within the spirit and scope of the disclosed subject matter as defined by the claims. In the drawings, which are not to scale, the same reference numerals are used throughout the description and in the drawing figures for components and elements having the same structure.

### DETAILED DESCRIPTION

In the following description, for purposes of explanation, numerous specific details are set forth to provide a thorough understanding of the inventive concept. In the interest of clarity, not all features of an actual implementation are described. Moreover, the language used in this disclosure has been principally selected for readability and instructional purposes and may not have been selected to delineate or circumscribe the inventive subject matter, resort to the claims being necessary to determine such inventive subject matter. Reference in this disclosure to “one embodiment” or to “an embodiment” or “another embodiment” means that a particular feature, structure, or characteristic described in connection with the embodiment is included in at least one embodiment of the disclosed subject matter, and multiple references to “one embodiment” or “an embodiment” or “another embodiment” should not be understood as necessarily all referring to the same embodiment.

This disclosure pertains to a GEOWP, which is a multi-well cell culture device for optimizing gas exchange and nutrient availability in a cell culture. Techniques disclosed herein look to apply the multi-well cell culture device to address the hypoxia problem in a traditional cell culture for several types of terminally differentiated cells. In certain embodiments, the GEOWP includes a GEOWP base, a GEOWP lid, and a GEOWP insert. In certain embodiments, the GEOWP base includes a plurality of alternating cell culture wells and reservoir wells. For example, the GEOWP base is made of plastic. Each of the plurality of cell culture wells has an elevated cell culture stage to hold a cell monolayer of adherent cells near the surface of a cell culture medium in which the adherent cells are cultured. Furthermore, each of the plurality of cell culture wells has a first plurality of openings above the elevated cell culture stage to be connected to a corresponding adjacent reservoir well which stores additional cell culture medium. In certain embodiments, the GEOWP lid includes a plurality of cylindrical protrusions to seal the plurality of reservoir wells. Thus, each of the plurality of reservoir wells is configured to hold a capped reservoir of the cell culture medium to limit evaporation effects. The cylindrical protrusions are configured to produce an airtight seal on the plurality of reservoir wells when the GEOWP lid is placed onto the base of the GEOWP base.

Furthermore, in certain embodiments, the GEOWP insert fits between the GEOWP base and the GEOWP lid to disconnect upper volumes and lower volumes of the plurality of cell culture wells in the GEOWP base. In particular, the GEOWP insert includes a plurality of downward protrusions to cover the first plurality of openings associated with the plurality of cell culture wells to disconnect volumes above and below the elevated cell culture stages associated with the plurality of cell culture wells. By using the GEOWP insert, the GEOWP may reduce the amount of reagent needed for transient cell manipulation by using a much smaller volume of reagent needed to produce the appropriate concentration in the small amount of cell culture medium above the cell monolayer. Thus, the GEOWP provides a cost-effective solution to solve the problem of hypoxia for adherent cells by optimizing gas exchange and cell culture conditions for the adherent cells. Additionally, the GEOWP may effectively reduce the workload associated with cell culture by reducing the number of medium changes required to maintain cells and making more efficient use of costly cell manipulation reagents.

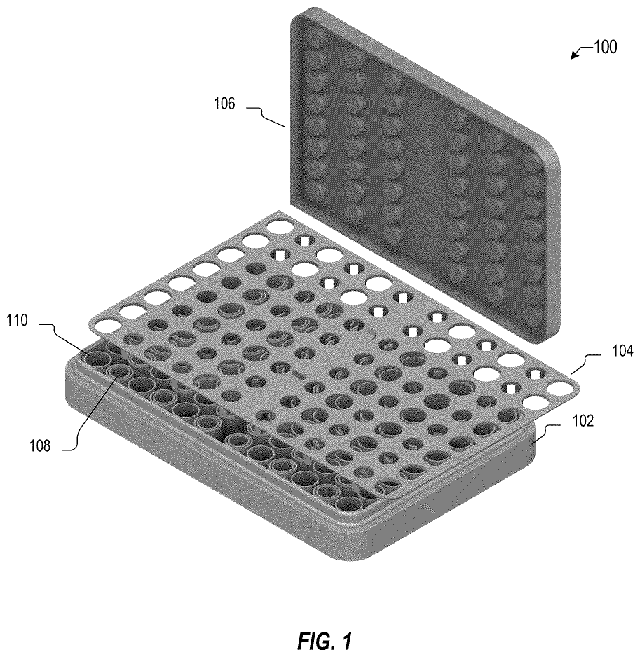

FIG. 1 illustrates a GEOWP 100 in accordance with one or more embodiments. In the illustrated embodiment, GEOWP 100 includes a base 102, an insert 104, and a lid 106 to improve cell culture conditions by minimizing hypoxia in cultured cells. In particular, the GEOWP base 102 includes a plurality of alternating cell culture wells 108 and reservoir wells 110 which are designed to optimize gas exchange and nutrient availability in a cell culture medium filled in GEOWP 100. In particular, each of the plurality of cell culture wells 108 includes an elevated cell culture stage which positions a plurality of cells in a cell monolayer closer to the surface of the cell culture medium, reducing the risk of hypoxia which is a state of oxygen deficiency. In some embodiments, cell culture is a potent tool for biological researchers to support cell growth and function in an artificial environment for different types of cells in a cell culture medium. In particular, a cell culture vessel is configured to be used as a container to culture a model system such as a plurality of cultured cells in a cell monolayer to resemble the system of interest closely. For example, the cell culture vessel is configured to culture skin cells in a dish under conditions like a native environment of the skin cells. Thus, the experimental results using the cell culture vessel may be translated to the system of interest, such as human skin. GEOWP 100 may mimic physiological conditions in the cell culture, thus, the cultured cells are produced in a model system which most closely emulates the system of interest. As a result, GEOWP 100 provides proper controls to mitigate the vast majority of false results due to hypoxia in traditional cell culture. Without properly managing the cellular microenvironment for the cell monolayer, the sensitivity of various experiments may be significantly limited.

In some embodiments, the plurality of cell culture wells 108 are configured to be used as cell culture vessels for cell monolayers of adherent cells and non-adherent cells. For the non-adherent cells, they are typically shaken to avoid accumulation of cells at the bottom of a cell culture vessel or they are cultured in a bioreactor. The non-adherent cells are not as prone to the hypoxia issue because they are not relegated to a surface at the maximum distance away from the fluid meniscus as is typically the case with the adherent cells.

In some embodiments, for the adherent cells, each cell monolayer is a layer of cells adhered to the surface of the cell culture vessel with a characteristic thickness of one cell. In some embodiments, the cell monolayers are the result of regular physiological functions in certain cell types, such as adherent cells, which adhere to one another in their usual biological contexts. The cell monolayers are the simplest and most common way of culturing adherent cells. For example, the plurality of cell culture wells 108 may be used to culture myocardial cells (heart muscle), which stick to one another and the cell culture vessel. Furthermore, the plurality of cell culture wells 108 can be configured to be used as cell culture vessels for other types of cell cultures, such as diffuse cell culture and three-dimensional (3D) cell cultures. For example, diffuse cell cultures are common for bacterial cells and blood cells. As another example, in 3D cell cultures, adherent cells may be grown on a frame, such as a hydrogel, to simulate tissue networks. In particular, the adherent cells may be in specific wells of the 3D cell cultures with specific geometries, such as conical, to enable organoid formation.

In some embodiments, the adherent cells may be used for the bulk of human cell culture applications in research. In particular, adherence allows for the construction of intricate and stable physiological structures and mechanical force generation. For instance, by treating an adherent cell culture with certain chemical solutions, one may loosen the cells enough to transfer them to different cell culture vessels. Furthermore, dead cells do not adhere which allows one to remove dead cells during medium changes without any trouble. Additionally, medium changes do not require centrifugation to separate the cells from the cell culture medium as would be the case with a diffuse cell culture. Instead, one can simply use a pump to remove the liquid in which the cells remain stuck to the plate and then replenish it gently. Furthermore, a cell monolayer thickness varies depending on cell type and culture conditions, but the cell monolayer thickness is often in the range of 2-5 micrometers (um).

In some embodiments, the adherent cells grow in a cell culture vessel which is cultured under the conditions of the living organisms, while attached to a surface, such as a flat circular surface, inside the cell culture vessel. For example, the cell culture vessel includes one or more cylindrical walls extending upwards around the circumference of a circular plate on which the cells sit to hold a fluid which acts as a cell culture medium to provide nutrients and other components to support cell growth and function under the conditions of the living organisms. Various cell culture mediums have been carefully designed to meet virtually all the demands of culturing different cells. These demands include a reduced carbon source to supply the cells with a substrate for oxidative phosphorylation (energy production), ions to facilitate transmembrane transport, and bicarbonate buffer to keep the pH level of the solution stable. Thus, the different types of cells may survive and replicate to produce more cells in the cell culture medium.

In some embodiments, different cell culture vessels ranging from large plastic dishes to well plates are configured to be used to supply the cell culture medium in the cell culture practice. For example, many cells can be grown easily in a plurality of large plastic dishes. As another example, a well plate may include a plurality of hollow cylinders to hold groups of cells separately, accommodating experiments with many varying conditions. With any cell culture practice, a major concern is the ability to keep somatic cells alive independently of their usual multicellular context to vastly expand the repertoire of experiments available to researchers. However, prior art cell culture practices indicate that the traditional cell culture medium used to cover adherent cells is sufficient to induce hypoxia in many terminally differentiated cell types, considering that pericellular oxygen improves the quality, reproducibility, and translatability of culture models. As a result, limited oxygen in the terminally differentiated cell types alters the metabolism and function of differentiated cells, leading to different types of malfunctional cellular functions or even cell deaths. Prior art practices implemented several solutions to solve the hypoxia issue at the cost of creating many other problems. For example, a cell culture vessel is designed to solve the hypoxia issue by using expensive materials or manufacturing methods which significantly increase the cost.

In some embodiments, each of the plurality of cell culture wells 108 is coupled with a corresponding reservoir well from the plurality of reservoir wells 110 via a channel to form a single reservoir system for the adherent cells. The volume above and below the elevated cell culture stage is hollow, allowing it to be filled with the cell culture medium. In some embodiments, a cell culture well has a first plurality of openings above an elevated cell culture stage to allow the exchange of nutrients in a cell culture medium between the volume above the elevated cell culture stage in the cell culture well and a corresponding reservoir well via a channel. Likewise, the cell culture well has a second plurality of openings below the elevated cell culture stage to allow the exchange of nutrients in the cell culture medium between the volume below the elevated cell culture stage in the cell culture well and the corresponding reservoir well via the channel. Thus, GEOWP 100 may provide additional cell culture medium, reduce evaporation effects for the plurality of cell culture wells 108, and optimize gas exchange and nutrient availability in cell culture base 102. As a result, GEOWP 100 provides a cost-effective, scalable solution which enhances gas exchange and minimizes the need for frequent medium changes, while also reducing reagent usage during cell manipulation processes.

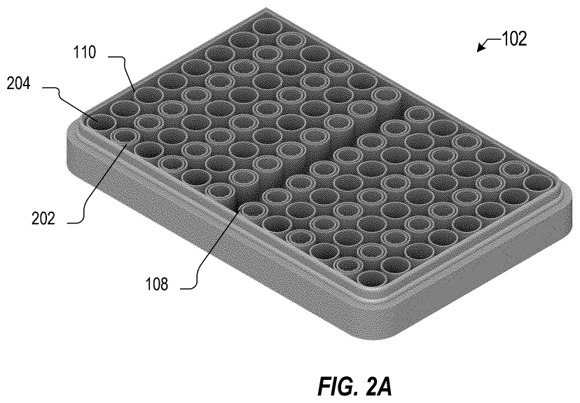

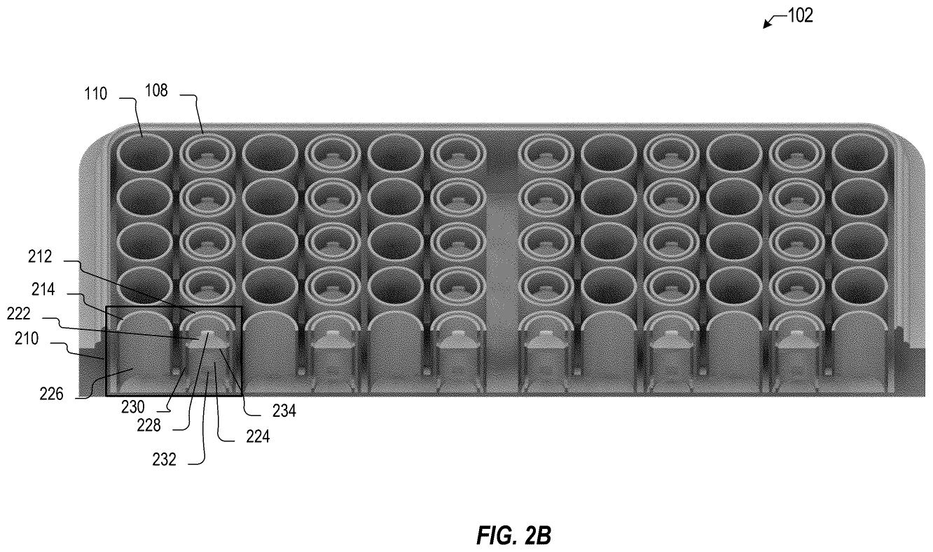

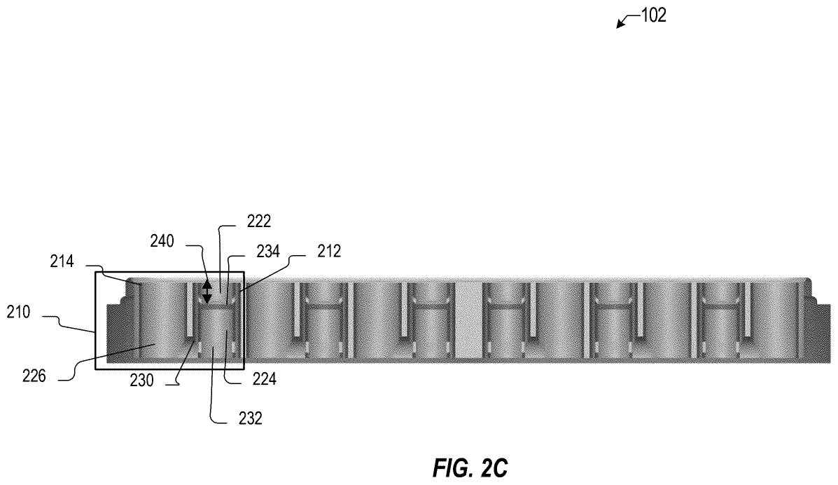

FIG. 2A illustrates a top-angle view of a GEOWP base 102 in accordance with one or more embodiments. The GEOWP base 102 contains a plurality of cell culture wells 108 and a plurality of reservoir wells 110. The plurality of cell culture wells 108 and the plurality of reservoir wells 110 are arranged in alternating order in the GEOWP base 102. As an example and not by way of limitation, the plurality of cell culture wells 108 are arranged in rows in the GEOWP base 102. Each of the plurality of cell culture wells 108 has two concentric cylinders 202 filled with a cell culture medium. Likewise, the plurality of reservoir wells 110 are arranged in rows in the GEOWP base 102. Each of the plurality of reservoir wells 110 has one cylinder 204 filled with the cell culture medium. In particular, each of the plurality of cell culture wells 108 is coupled via a channel to a corresponding reservoir well from the plurality of reservoir wells 110.

FIG. 2B illustrates a top-front angle view of a GEOWP base 102 in accordance with one or more embodiments. In GEOWP base 102, a plurality of cell culture wells 108 and a plurality of reservoir wells 110 form a plurality of subgroups 210 aligned in multiple columns. Each of the plurality of subgroups 210 includes a corresponding cell culture well 212 and a corresponding reservoir well 214 which are connected via channel 230. Thus, each subgroup 210 is configured to be used as cell culture equipment to contain a cell culture medium for a type of cell, such as adherent cells. In particular, cell culture well 212 includes an elevated cell culture stage 234 to hold cultured cells near the surface of the cell culture medium in which they are cultured. In some embodiments, based on Fick's first law of diffusion, elevated cell culture stage 234 provides a solution to the hypoxia problem in traditional adherent cell culture by reducing the distance between the cell monolayer and the surface of the cell culture medium where gas exchange occurs.

In some embodiments, cell culture well 212 is coupled to a capped reservoir, such as reservoir well 214, via channel 230. For the traditional low-medium-height cell culture, reducing the cell culture medium height introduces additional evaporation and nutrient depletion concerns. Alternatives to the low-medium-height cell culture do exist, such as well plates with gas-permeable bases. However, they are far more costly to buy presumably due to the increased cost of manufacturing. GEOWP base 102 is configured to use elevated cell culture stage 234 in conjunction with the capped reservoir, such as reservoir well 214, that is attached to the cell culture well 212 by channel 230 to make the low-medium-height cell culture more feasible. For example, GEOWP base 102 is made of plastic, such as polystyrene, via injection molding to provide a solution for the economic and scalable production of well plates. As another example, elevated cell culture stage 234 may be made of glass if confocal microscopy is to be used.

In some embodiments, cell culture well 212 includes two hollow concentric cylinders 202 (referring to FIG. 2A) filled with the cell culture medium. In particular, the volume cell culture well 212 is divided into an upper volume 222 and a lower volume 224 by elevated cell culture stage 234. Likewise, reservoir well 214 includes a hollow cylinder 204 (referring to FIG. 2A) having a volume 226 filled with the cell culture medium. In some embodiments, reservoir well 214 is configured to store the additional cell culture medium in an airtight fashion. The volume, such as the upper volume 222 of the cell culture well 212, the lower volume 224 of the cell culture well 212, and the volume 226 of the reservoir well 214, in a reservoir system may be increased as desired without adversely affecting gas exchange thus allowing for longer periods of undisturbed cell culture.

In some embodiments, cell culture well 212 includes a first plurality of openings 228 above elevated cell culture stage 234 to be connected to a corresponding adjacent reservoir well 214 which stores additional cell culture medium. Likewise, cell culture well 212 includes a second plurality of openings 232 below elevated cell culture stage 234 to be connected to the corresponding adjacent reservoir well 214. Thus, the upper volume 222 above elevated cell culture stage 234 is linked to the lower volume 224 below elevated cell culture stage 234 via the first plurality of openings 228 and the second plurality of openings 232. In some embodiments, the size of the first plurality of openings 228 and the second plurality of openings 232 is large enough to enable sufficient mixing to facilitate liquid diffusion. For example, the size of the first plurality of openings 228 and the second plurality of openings 232 is no smaller than 2 mm² in area.

FIG. 2C illustrates a front view cross-section view of a GEOWP base 102 in accordance with one or more embodiments. In some embodiments, GEOWP base 102 is configured to use multiple static components for a cell culture well 212 and a reservoir well 214 in a subgroup 210 to optimize gas exchange in the culturing of adherent cells. Firstly, GEOWP base 102 raises the cell monolayer to within a predetermined distance, such as 2 mm, from the surface of the cell culture medium to avoid inducing hypoxia in the cells. Secondly, assuming the cell culture well 212 and the reservoir well 214 have equal volume, the cultured cells may have access to approximately twice as much cell culture medium as they would in a traditional cell culture vessel.

In some embodiments, elevated cell culture stage 234 on which the cells are cultured is not at the bottom of the cell culture well 212 but is elevated such that the cell monolayer resides about 2 mm from the surface of the cell culture medium. The lower volume 224 below elevated cell culture stage 234 in the cell culture well 212 is hollow enabling it to fill with the cell culture medium. For example, the hollow space, such as lower volume 224, beneath elevated cell culture stage 234 is connected to the space, such as upper volume 222, above the cell monolayer by a small tube via the second plurality of opening 232, which enables the exchange of nutrients between the upper volume 222 of cell culture well 212 and the lower volume 224 of cell culture well 212.

In some embodiments, there are several constraints on the dimensions of the cell culture well 212 when the cell culture well 212 is scaled up or down for a specific product. For example, the cell culture well 212 includes a distance 240 from elevated cell culture stage 234 to the rim of the cell culture well 212 which is greater than 2 mm to allow 2 mm medium coverage plus some additional clearance. As another example, there is a small gap, such as no less than 1 mm, between the rim of cell culture well 212 and the bottom surface of the GEOWP lid 106 (referring to FIG. 1) to allow for gas exchange between cell culture well 212 and the local atmosphere.

In some embodiments, reservoir well 214 is a separate well near cell culture well 212. Reservoir well 214 is a hollow cylinder much like cell culture well 212 but does not contain a cell stage thus making it a single unobstructed hollow volume 226 which is filled with cell culture medium. The volume 226 inside reservoir well 214 is linked to the lower volume 224 under elevated cell culture stage 234 in cell culture well 212 by the second plurality of opening 232 and a small channel 230 that runs between them on the base of GEOWP base 102. The amount of nutrients the cultured cells have access to may be further increased by increasing the volume 226 of the reservoir well 214 relative to the area of elevated cell culture stage 234. The same may also be achieved by increasing the lower volume 224 of the space under elevated cell culture stage 234 without increasing the area of the cell culture stage 234.

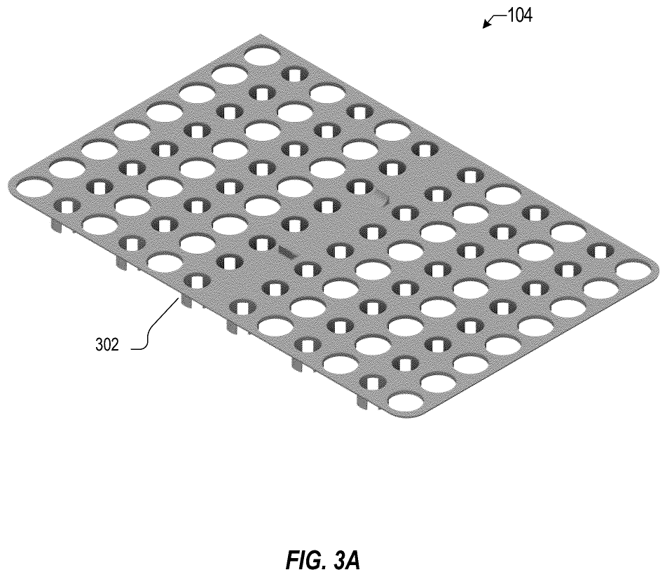

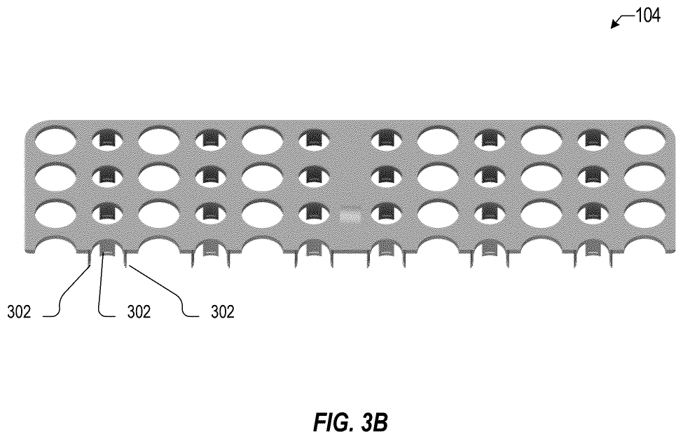

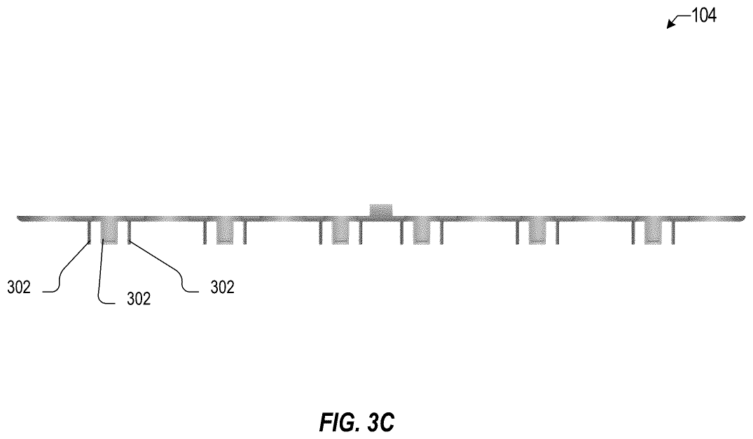

FIG. 3A illustrates a top-angle view of a GEOWP insert 104 in accordance with one or more embodiments. In particular, GEOWP insert 104 fits between GEOWP base 102 (referring to FIG. 1) and GEOWP lid 106 (referring to FIG. 1) for a cell culture vessel. GEOWP insert 104 has a plurality of downward protrusions 302 to cover the first plurality of openings 228 (referring to FIG. 2B) associated with the plurality of cell culture wells 108 (referring to FIG. 2B) to disconnect volumes above and below elevated cell culture stages 234 (referring to FIG. 2B), such as upper volume 222 (referring to FIG. 2B) and lower volume 224 (referring to FIG. 2B), associated with the plurality of cell culture wells 108 (referring to FIG. 2B). Disconnecting these two spaces enables a user to seed adherent cells onto elevated cell culture stage 234 (referring to FIG. 2B) without the cells escaping to other chambers of the cell culture vessel. After sufficient time for seeded cells to adhere to elevated cell culture stage 234 (referring to FIG. 2B), the cell culture medium may be aspirated, and GEOWP insert 104 may be removed to use the cell culture vessel for its intended benefits.

FIG. 3B illustrates a top front cross-section view of a GEOWP insert 104 in accordance with one or more embodiments. FIG. 3C illustrates a front angle cross-section view of a GEOWP insert 104 in accordance with one or more embodiments. In this example, GEOWP insert 104 includes three downward protrusions 302 to cover three openings associated with a cell culture well of a reservoir system. GEOWP insert 104 allows a user to save on reagents needed for transient cell manipulations, such as transfection or viral infection. Without using GEOWP insert 104, the user may have to use a volume of reagent appropriate for the entire volume of the reservoir system. With using GEOWP insert 104, the user may use a much smaller volume of reagent which is needed to produce the appropriate concentration in the small amount of cell culture medium above the cell monolayer. Furthermore, the user may remove GEOWP insert 104 after a sufficient incubation period as the growth of the cell monolayer is stable.

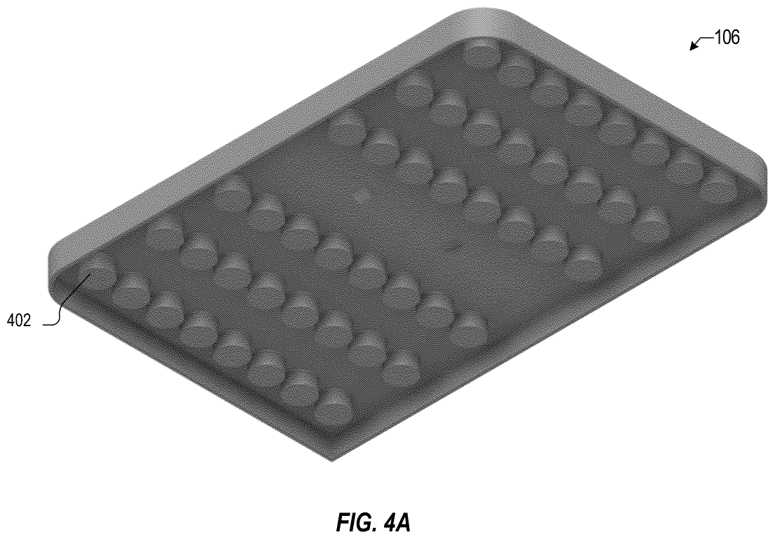

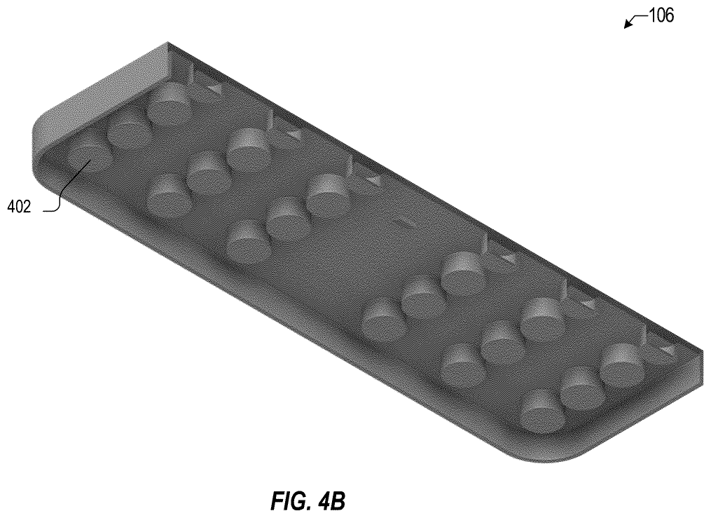

FIG. 4A illustrates a bottom-angle view of a GEOWP lid 106 in accordance with one or more embodiments. FIG. 4B illustrates a bottom-angle cross-section view of a GEOWP lid 106 in accordance with one or more embodiments. GEOWP lid 106 includes a plurality of cylindrical protrusions 402 to seal a plurality of reservoir wells 110 (referring to FIG. 2B) of a reservoir system. In particular, GEOWP lid 106 is configured to mitigate the increased risk of evaporation effects due to there being a small volume of cell culture medium above the cell monolayer. For example, GEOWP lid 106 contains cylindrical protrusions 402 which produce an airtight seal on the plurality of reservoir wells 110 (referring to FIG. 2B) when GEOWP lid 106 is placed onto the base of a well plate. The size of the cylindrical protrusions 402 is large enough to sufficiently seal the plurality of reservoir wells 110 (referring to FIG. 2B) of the reservoir system.

In some embodiments, GEOWP lid 106 is configured to seal the reservoir system to minimize evaporation and maintain a constant cell culture medium level. Thus, evaporation becomes a closed process in each of the plurality of reservoir wells 110 (referring to FIG. 2B). GEOWP lid 106 may disable the cell culture medium height in the corresponding reservoir well from decreasing significantly. In some embodiments, the volume in a cell culture well is linked to the volume in a corresponding reservoir well. If the cross-sectional area of both wells is the same, the rate of medium height decrease in the cell culture well may be decreased by approximately half. The rate of evaporation-induced medium height decrease may be further diminished by increasing the ratio of the cross-sectional area of the reservoir well to the cross-sectional area of the cell culture well. However, the cell culture well may not be capped because producing an air-tight seal above the cell monolayer may limit gas exchange with the external environment and starve the cells of oxygen. The rate of evaporation from the cell culture well may be further decreased by filling the space between wells with fluid, such as water or phosphate-buffered saline, which will saturate the local atmosphere with water molecules and increase the rate of condensation of the water molecules back into the fluid state.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described herein are to be taken as examples of embodiments. Elements and materials may be substituted for those illustrated and described herein, parts and processes may be reversed or omitted, and certain features of the embodiments may be utilized independently, all as would be apparent to one skilled in the art after having the benefit of this description of the embodiments. Changes may be made in the elements described herein without departing from the spirit and scope of the embodiments as described in the following claims. Headings used herein are for organizational purposes only and are not meant to be used to limit the scope of the description.

It will be appreciated that the processes and methods described herein are example embodiments of processes and methods that may be employed in accordance with the techniques described herein. The processes and methods may be modified to facilitate variations of their implementation and use. The order of the processes and methods and the operations provided may be changed, and various elements may be added, reordered, combined, omitted, modified, and so forth. Portions of the processes and methods may be implemented in software, hardware, or a combination of software and hardware. Some or all of the portions of the processes and methods may be implemented by one or more of the processors/modules/applications described here.

As used throughout this application, the word “may” is used in a permissive sense (that is, meaning having the potential to), rather than the mandatory sense (that is, meaning must). The words “include,” “including,” and “includes” mean including, but not limited to. As used throughout this application, the singular forms “a,” “an,” and “the” include plural referents unless the content clearly indicates otherwise. Thus, for example, reference to “an element” may include a combination of two or more elements. As used throughout this application, the term “or” is used in an inclusive sense, unless indicated otherwise. That is, a description of an element including A or B may refer to the element including one or both of A and B. As used throughout this application, the phrase “based on” does not limit the associated operation to being solely based on a particular item. Thus, for example, processing “based on” data A may include processing based at least in part on data A and based at least in part on data B, unless the content clearly indicates otherwise. As used throughout this application, the term “from” does not limit the associated operation to being directly from. Thus, for example, receiving an item “from” an entity may include receiving an item directly from the entity or indirectly from the entity (for example, by way of an intermediary entity). Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. In the context of this specification, a special purpose computer or a similar special purpose electronic processing/computing device is capable of manipulating or transforming signals, typically represented as physical, electronic or magnetic quantities within memories, registers, or other information storage devices, transmission devices, or display devices of the special purpose computer or similar special purpose electronic processing/computing device.

At least one embodiment is disclosed and variations, combinations, modifications of the embodiment(s), or features of the embodiment(s) made by a person having ordinary skill in the art are within the scope of the disclosure. Alternative embodiments that result from combining, integrating, or omitting features of the embodiment(s) are also within the scope of the disclosure. Where numerical ranges or limitations are expressly stated, such express ranges or limitations may be understood to include iterative ranges or limitations of like magnitude falling within the expressly stated ranges or limitations (for example, from about 1 to about 10 includes, 2, 3, 4, etc.; greater than 0.10 includes 0.11, 0.12, 0.13, etc.). The use of the term “about” (or its variants) means ±10% of the subsequent number, unless otherwise stated.

Use of the term “optionally” with respect to any element of a claim means that the element is required, or alternatively, the element is not required, both alternatives being within the scope of the claim. Use of broader terms such as comprises, includes, and having may be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise.

Many other embodiments will be apparent to those of skill in the art upon reviewing the above description. The scope of the subject matter of the present disclosure therefore should be determined with reference to the appended claims, along with the full scope of equivalents to which such claims are entitled. In the appended claims, the terms “including” and “in which” are used as the plain-English equivalents of the respective terms “comprising” and “wherein.”

What is claimed is:

1. A multi-well cell culture device for optimizing gas exchange and nutrient availability in a cell culture, comprising:
a Gas Exchange Optimized Well Plate (GEOWP) base, the GEOWP base comprising a plurality of alternating cell culture wells and reservoir wells, each of the plurality of cell culture wells having an elevated cell culture stage to culture adherent cells and a first plurality of openings above the elevated cell culture stage to be connected to a corresponding adjacent reservoir well which stores additional cell culture medium;
a GEOWP lid having a plurality of cylindrical protrusions to seal the plurality of reservoir wells; and
a GEOWP insert which fits between the GEOWP base and the GEOWP lid, the GEOWP insert having a plurality of downward protrusions to cover the first plurality of openings associated with the plurality of cell culture wells to disconnect volumes above and below the elevated cell culture stages associated with the plurality of cell culture wells.

2. The multi-well cell culture device of claim 1, wherein each of the plurality of cell culture wells has a second plurality of openings below the elevated cell culture stage.

3. The multi-well cell culture device of claim 2, wherein a volume above the elevated cell culture stage is linked to a volume below the elevated cell culture stage via the first plurality of openings above the elevated cell culture stage and the second plurality of openings below the elevated cell culture stage.

4. The multi-well cell culture device of claim 3, wherein:
each of the plurality of reservoir wells is configured to store the additional cell culture medium in an airtight fashion, and
each of the plurality of reservoir wells is configured to be connected to a corresponding cell culture well through a channel.

5. The multi-well cell culture device of claim 1, wherein each of the plurality of cell culture wells comprises two concentric hollow cylinders.

6. The multi-well cell culture device of claim 1, wherein each of the plurality of reservoir wells comprises a hollow cylinder.

7. The multi-well cell culture device of claim 1, wherein the plurality of reservoir wells uses multiple static components to optimize gas exchange in the culturing of adherent cells.

8. The multi-well cell culture device of claim 1, wherein a cell monolayer of the adherent cells which adhere to the elevated cell culture stage is raised to within a predetermined distance from the surface of the cell culture medium to avoid inducing hypoxia in the cells.

9. The multi-well cell culture device of claim 8, wherein the cell monolayer of the adherent cells resides only about 2 mm from the surface of the cell culture medium.

10. The multi-well cell culture device of claim 1, wherein the GEOWP base is made of plastic.
